(19) United States
(12) Patent Application Publication
(10) Pub. No.: US 2005/0267334 A1
(43) Pub. Date: Dec. 1, 2005

(54) Fluid propelled endoscope

(76) Inventors: Christopher P. Swain, London (GB); Charles A. Mosse, London (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/115,748

(22) Filed: Apr. 27, 2005

(30) Foreign Application Priority Data
Apr. 28, 2004 (GB) UK0409485.0

(51) Int. Cl. A61B 1/015

(52) U.S. Cl. 600/156; 600/159; 600/114

(57) ABSTRACT

An endoscope, such as a colonoscope, is provided for introduction into and along a passageway in a human or animal body in a desired direction of travel. The endoscope is disclosed in association with a fluid propulsion apparatus which can be employed for effecting introduction and advancement of the endoscope.

This application claims priority to and incorporates by reference UK Patent Application UK 0409485.0 “Fluid Propelled Endoscope” filed Apr. 28, 2004.

The present invention relates to fluid propelled endoscopes, and to methods for using such endoscopes. The invention is particularly directed to endoscopes for use in the colon, i.e. colonoscopes, and more particularly to colonoscopes for use in the human colon. The following description focuses primarily on such colonoscopes. It is to be understood, however, that the invention is also applicable to other types of endoscope, for example, for use in the small bowel, and to endoscopes, whether or not they are colonoscopes, for use in the colons of non-humans.

### BACKGROUND OF THE INVENTION

One of the main difficulties in inserting a colonoscope into the colon is that the colon is an elastic tube that follows a tortuous path. As the colonoscope is pushed around a bend it does not always slide smoothly along the colon wall, but frequently catches on it, so that as the colonoscope is pushed further in, the colon is stretched and a loop is formed. In view of this, it would be desirable to provide traction at or near the tip of the colonoscope, to pull the colonoscope and so obviate, or at least lessen, the need for pushing.

One method which has been described in the art (see U.S. Pat. No. 4,735,501) for exerting traction on the tip region of an endoscope involves the use of fluid. Thus, in U.S. Pat. No. 4,735,501, various devices generically referred to as borescopes are described which have a fluid conduit along the length thereof, with an outlet at the distal end directed oppositely to the intended direction of insertion of the borescope. Fluid expelled from that outlet produces a reaction force which urges the borescope in its intended direction of insertion.

One reason why fluid propulsion is attractive for endoscopy is that very little mechanism is required inside the patient, and complex components, such as a pump for providing pressurised fluid, and a control system, can all be placed well away from the patient, where size is not a problem. However, there is a problem in using fluid propulsion in endoscopy, which is that, in practice, the thrust that can be produced is not very large. There are two reasons for this. Firstly, the flow rate is limited by the need not to overload the colon with water. The second is that the velocity of the fluid should not be so high that the fluid jet causes significant discomfort to the patient.

### SUMMARY OF THE INVENTION

According to one embodiment of the invention, there is provided an endoscope for introduction into and along a passageway in a human or animal body in a desired direction of travel. The endoscope can include fluid propulsion apparatus for effecting said introduction, with the fluid propulsion apparatus comprising a conduit having an inlet for fluid under pressure and at least one outlet for expelling the fluid in a direction at least partly opposed to the desired direction of travel of the endoscope, wherein the outlet, or each of the outlets, can be an atomising nozzle.

According to another embodiment of the invention there is provided an endoscope for introduction into and along a passageway in a human or animal body in a desired direction of travel, the endoscope having fluid propulsion apparatus for effecting introduction. The fluid propulsion apparatus can comprise a conduit having an inlet for fluid under pressure and at least one outlet for expelling fluid in a direction at least partly opposed to the desired direction of travel of the endoscope. The conduit can be adapted to be movable lengthwise of the endoscope, and can be constrained to substantially follow the path of the endoscope. The invention can provide a method of using such an endoscope, in which:
(a) the conduit is moved a certain direction in the desired direction of travel;
(b) the endoscope is allowed to move in the said desired direction; and
(c) steps (a) and (b) are repeated until the endoscope has been introduced to the desired extent.

In another embodiment, the invention provides a method of introducing an endoscope into and along a passageway in a human body, the endoscope having fluid propulsion apparatus for effecting the introduction, wherein the fluid propulsion apparatus comprises a conduit having an inlet for fluid under pressure and at least one outlet, wherein a guidewire is introduced into and along said passageway, and wherein the endoscope is caused to travel over said guidewire, in the desired direction of travel, by expelling fluid from said outlet in a direction at least partly opposed to the desired direction of travel. The guidewire may, for example, be introduced into and along said passageway by a method as described in UK Patent Application No. 0307715.3, corresponding to co-pending U.S. patent application Ser. No. 10/409,270 (Swain et al) and PCT/US2004/009982 (published as WO2004/089456), the contents of which are incorporated herein by reference.

The present invention additionally provides an endoscope adapted for use in the method just defined, where a guidewire is introduced as a preliminary step, the endoscope comprising an elongate main body having a leading end and a proximal end, and a guidewire receiving element exterior to the main body, at or adjacent the distal end thereof, for defining an opening through which the guidewire can pass. The guidewire receiving element is preferably in the form of a short tube, i.e. a tube short compared to the length of the main body, secured to, or integral with, the main body.

In yet another embodiment, the present invention provides an endoscope for introduction into and along a passageway in a human or animal body in a desired direction of travel, the endoscope having fluid propulsion apparatus for effecting said introduction, said fluid propulsion means comprising a conduit having an inlet for fluid under pressure and at least one outlet for expelling said fluid in a direction at least partly opposed to the desired direction of travel of the endoscope, wherein the endoscope has: (a) a mass per unit length, over a majority of its length, of not more than about 1 gram/cm; and/or (b) a diameter of less than about 6 mm over a majority of its length.

In a yet further embodiment, the invention provides a colonoscope for introduction into and along a human or animal colon in a desired direction of travel, the endoscope having fluid propulsion means for effecting said introduction, said fluid propulsion means comprising a conduit having an inlet for fluid under pressure and at least one outlet for expelling said fluid in a direction at least partly opposed to the desired direction of travel of the endoscope.

Where a fluid is referred to herein it is to be understood that this is preferably a liquid, and that the liquid is preferably aqueous. The liquid would normally be water, or an aqueous liquid which is isotonic with the liquid expected to be found in the passageway into which the endoscope is being introduced. It may be desirable to add a biologically acceptable anti-foaming agent or other additive. Also, it may be desirable to warm the water to body temperature.

### BRIEF DESCRIPTION OF THE DRAWINGS

Some embodiments of the invention will now be described, with reference to the accompanying drawings, in which:

FIG. 1 shows an embodiment of colonoscope according to the present invention in the process of being introduced into, and passed along, the colon;

FIG. 2 is a longitudinal section through an atomiser head for use at the distal end of an embodiment of the invention;

FIG. 2a is a cross-sectional view on line II-II in FIG. 2, but omitting the central tube;

FIG. 3 shows an endoscope according to the present invention mounted on a guidewire for travel along that guidewire;

FIGS. 4a and 4b show two possible arrangements for supplying liquid under pressure to the endoscope.

### DETAILED DESCRIPTION

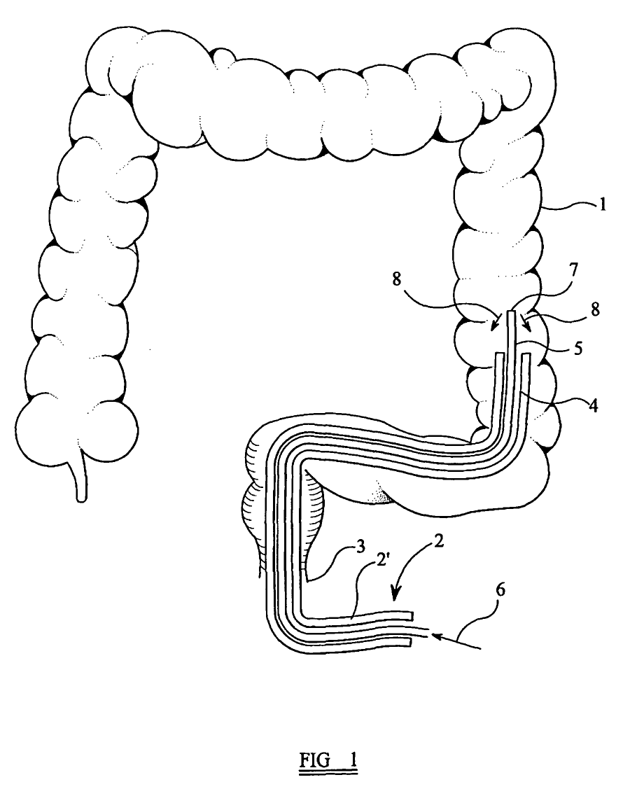

Referring now in more detail to FIG. 1, FIG. 1 shows a colon 1 and an endoscope 2 which, in the position shown, has been introduced through the anus 3 so that the leading end (distal end) of the endoscope is located part way along the length of the colon. The endoscope 2 has a flexible, elongate main body 2′ with a biopsy channel 4 extending along the length thereof. It will be understood that the endoscope is shown only diagrammatically, and that the diameter of the biopsy channel would in practice be much smaller in relation to the diameter of the endoscope body than is shown in the drawing. Also, for simplicity, the optics of the endoscope have been omitted, as have the controls which are positioned at the proximal end of the endoscope. The endoscopes and controls can be entirely conventional, and form no part of the present invention.

A flexible conduit 5 passes through the biopsy channel 4, emerging from the channel at both ends. The conduit can be made of, for example, a medical-grade plastics material and is longitudinally slideable within the biopsy channel. The upstream end of the conduit 5 is connected to a liquid supply, indicated by the arrow 6, for supplying liquid under pressure. The distal end 7 of the conduit 5 is closed to prevent liquid emerging therefrom, and outlets are formed laterally in the conduit through which jets of liquid 8 emerge. The embodiment of FIG. 1 has four such outlets, arranged at 90° to one another around the distal end region of the conduit (two jets of liquid 8 are visible in FIG. 1). However, it is to be understood that there might be only one, two or three outlets, or there might be more than four. The outlets are so formed that the jets of liquid, as they leave the outlets, have a component directed lengthwise of the conduit 5 in a direction away from its distal end and towards its proximal end. In the embodiment illustrated in FIG. 1 the jets are angled backwards, i.e. they not only have the component just defined but also a component at right angles to the length of the conduit and, as illustrated, towards the adjacent part of the colon wall. It is to be understood, however, that the outlets could be so formed that the jets were directed parallel to the length of the conduit in the distal end region, i.e. they had no component directed towards the adjacent part of the colon wall.

A description will now be given of the way in which the endoscope of FIG. 1 is advanced further into the colon, starting from the position shown in the figure. Initially, the physician carrying out the endoscopy holds the proximal end of the endoscope main body 2′ and the adjacent portion of the conduit 5, so that they cannot move longitudinally with respect to one another at that point. Liquid under pressure is then supplied to the proximal end of the conduit 5 from the above-mentioned liquid supply and emerges from the distal end of the conduit in the form of liquid jets 8. These jets produce a reaction force on the distal portion of the conduit which cause the conduit to move forwards, further into the colon. Although the conduit 5 is slideable within the biopsy channel 4 of the main body 2′, it has surprisingly been observed that forward movement of the conduit 5 is accompanied by forward movement of the endoscope main body. Without being limited by theory, it is believed that this is due not to friction between the conduit and main body but to the tendency of the main body to try to straighten in response to forward movement of the leading end of the conduit 5, which of course tends to straighten that conduit. In this way, the liquid jets 8 provide what appears to be, at least in substance, equivalent to a traction force on the distal end of the conduit main body 2′, which is what is desired.

The endoscope main body 2′ can be of the type which has a steerable tip. Such articulating endoscopes are known in the art. Altering the angle of that tip with respect to the remainder of the body has the effect of altering the direction in which the distal end portion of the conduit points, and therefore altering the direction in which the liquid jets 5 exert their propulsion force. In this way the leading end of the endoscope can be steered along the tortuous path which is followed by the colon. If the endoscope employed does not have such a steerable tip, it may be desirable to prevent the distal tip of the endoscope from abutting against the wall of the colon or into a diverticulum, so that further thrust from the tip would not advance the endoscope. Accordingly, it may be desirable to provide an apparatus for altering the direction of the jets 8 when a non-articulating endoscope is employed, or provide an apparatus that alters the direction of the force provided by those jets. For instance, it may be desirable to provide independent control of the velocity and/or volumetric flow rate through individual ones of a plurality of jets when an endoscope without a steerable tip is employed. Alternatively, such independent control can be used with an endoscope having a steerable tip.

As mentioned above, when using jet propulsion in endoscopy it is desirable that the velocity of the fluid is not so high that it causes significant discomfort to the patient. With this in mind, the present invention provides, in one of its aspects, an endoscope in which fluid propulsion is provided by means of one or more atomising nozzles. Surprisingly, it has been found that atomising the liquid to produce a spray causes very little reduction in the efficiency of propulsion, while potentially providing the benefit of reduced risk of discomfort.

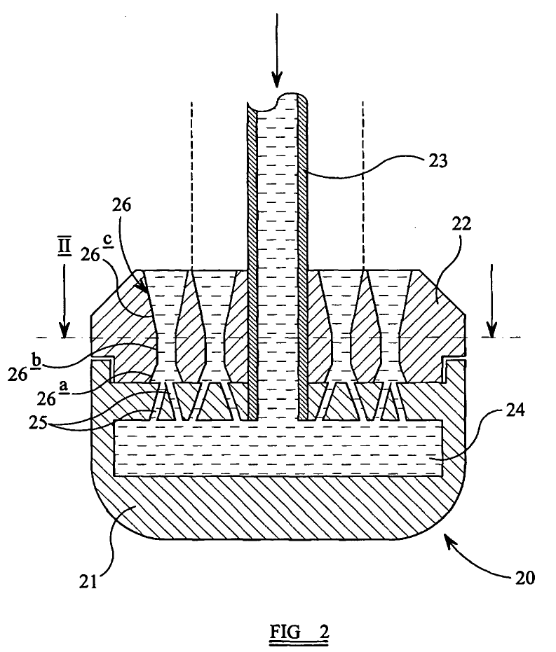

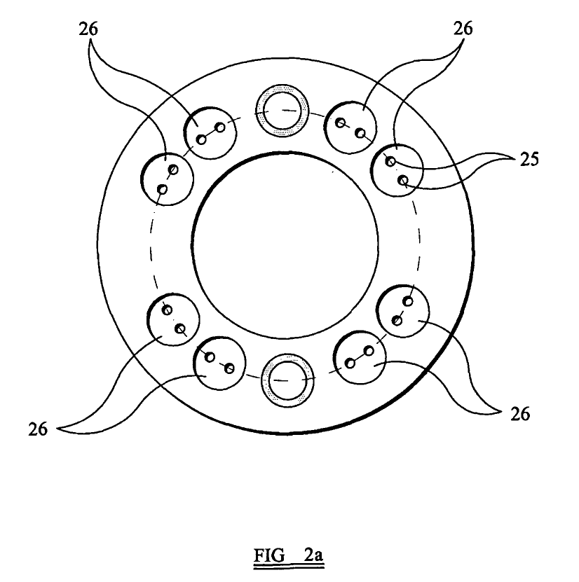

An atomiser head for use in this aspect of the invention is shown in FIG. 2. The head 20 comprises a distal body portion 21 and a proximal body portion 22 within which are formed cavities which enable atomised liquid jets to be formed. In FIG. 2 dense hatching represents liquid, and sparse hatching represents spray. Liquid enters the head through a tube 23 which leads into a plenum chamber 24. From the chamber 24 liquid passes through a plurality of pairs of bores 25, the bores of each pair being angled so that they meet at their downstream ends. The liquid travelling through one bore of the pair collides at its downstream end with liquid travelling through the other bore of the pair, and this collision causes atomisation, i.e. the break up of the liquid into a fine spray of droplets. It has been found that disposing the bores at angles of 20° to the longitudinal axis of the head, i.e. so that the bores of a given pair converge at an angle of 40°, produces good atomisation. In the particular experiment from which this result was derived, the bores were drilled in the member 21 with a 0.52 mm drill bit. It is to be understood, however, that these dimensions are given by way of example, and that other dimensions may be used.

Each pair of bores leads into a respective nozzle 26 which has a convergent upstream portion 26a, a constant diameter throat portion 26b and a divergent outlet portion 26c. The atomised jet emerges from the downstream end of the outlet portion 26c. In the embodiment shown in FIG. 2 there are eight nozzles 26 arranged as shown in FIG. 2a. It is to be understood, however, that there might be more nozzles or fewer nozzles. It is also to be understood that although the nozzles 26 are shown in FIG. 2 as pointing directly backwards, i.e. their direction of flow has no net component directed towards the adjacent walls of the colon, the nozzles might be directed in some fashion other than as shown in FIG. 2, for example they might be directed to produce fluid jets angled as shown in FIG. 1. It is also to be understood that many other types of atomising nozzles are known in the atomising art, and that such other nozzles might be used instead of a nozzle of the type illustrated.

In one aspect of the invention the endoscope is guided by means of a pre-installed guidewire. A suitable guidewire is disclosed in above mentioned UK Patent Application No. 0307715.3, corresponding to U.S. patent application Ser. No. 10/409,270, incorporated herein by reference. It is also to be understood that it is not essential that the guidewire should be pre-installed by the method described in UK Patent Application No. 0307715.3, and that any suitable alternative method for pre-installation of a guidewire might be used instead, and that alternative guidewire structures may also be useful.

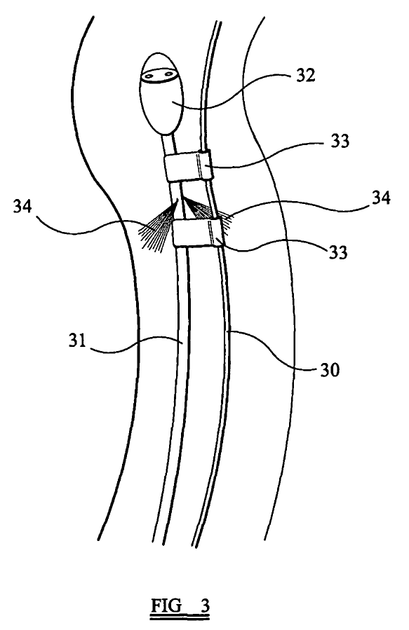

With the guidewire pre-installed, the endoscope can be threaded onto the portion of the guidewire protruding from the patient, and then caused to advance along the colon by fluid propulsion. FIG. 3 shows this process in the case of one suitable embodiment of endoscope. FIG. 3 shows a pre-installed guidewire 30 and, running generally parallel thereto, a flexible conduit 31 with a small endoscope head 32 on the leading end thereof. The head 32 may, for example, comprise an imaging camera suitable for viewing and/or recording images of the tissue within the GI tract of the patient. At least one length of tube 33 is attached to the conduit 31 and is slideable over the guidewire 30. Two lengths of tube are shown, but there may be three or more or there may be only one. The conduit 31 has backwardly pointing nozzles from which emerge propulsive jets 34, which operate like the jets 8 in FIG. 1. In addition to providing a passageway for propulsive liquid to travel to the nozzles, the conduit could also carry electric wires to enable power to be supplied to the head or electric signals conducted from the head. The conduit 31 and head 32 together constitute a lightweight endoscope (the conduit preferably has a mass of not more than about 1 g/cm) which, except for the head, and thus over a majority of its length, is of low diameter (the conduit preferably has a diameter of less than about 6 mm).

The use of a pre-installed guidewire is particularly useful where the endoscope is very lightweight (not more than 1 g/cm) and/or of low diameter (less than 6 mm). Providing a pre-installed guidewire can provide the advantage that there is no need to provide the endoscope itself with any means for steering it (such as with a steerable tip), and this in turn means that the endoscope can be a relatively inexpensive device, and/or can be provided or designed to include only those components and/or features that are desired for a particular procedure. For instance, an endoscope without a steerable tip but which includes imaging optics and a fluid conduit with at least one propulsion outlet at its distal end could be employed.

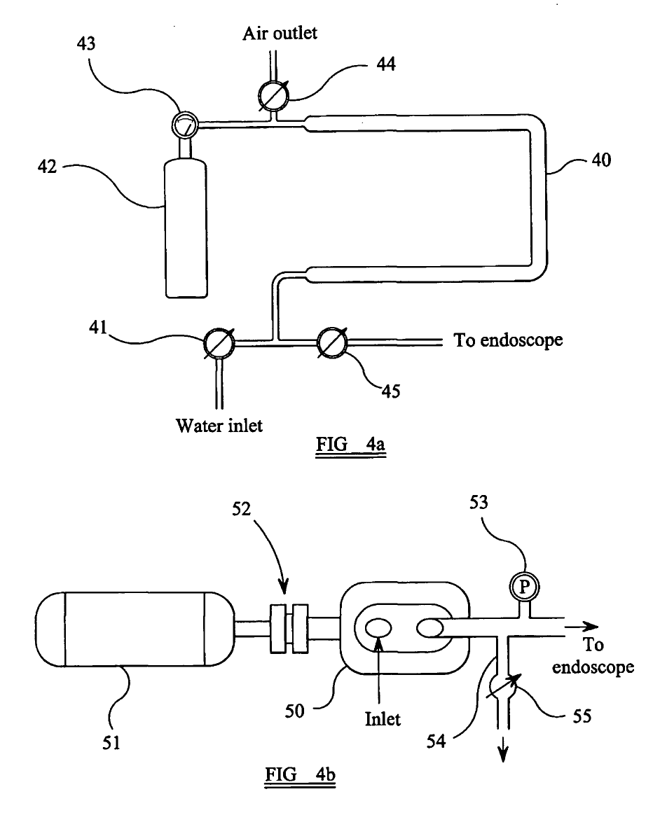

FIGS. 4a and 4b show alternative arrangements for delivering pressurised liquid (such as for instance water) to an endoscope of the present invention. The arrangement shown in FIG. 4 can be used to provide water at constant pressure. The arrangement comprises a reservoir 40 made from a length (5 m) of 2 inch diameter copper pipe. Water is supplied to the reservoir from a water inlet via an inlet valve 41. The water in the reservoir is pressurised to a suitable pressure, which depends on the internal diameter of the tube delivering the liquid, but which may, for example, be 6-8 Bar, by means of a cylinder 42 of pressurised nitrogen, connected to the reservoir 40 via a pressure regulator 43. The gas supply is capable of being connected to the outside atmosphere, to vent it, via a valve 44. Water under pressure is supplied to the endoscope via an outlet valve 45.

It may be desirable to use a supply of liquid at constant volumetric flow rate, rather than at constant pressure. The supply arrangement shown in FIG. 4b can be employed to provide such a constant volumetric flow rate. FIG. 4b shows a pump 50 designed to supply liquid at a constant volumetric flow rate, the pump being driven by an electric motor 51 via a flexible coupling 52. The pump has a water inlet, and has a water outlet connected to the endoscope. The pressure of the water being supplied is monitored by a pressure gauge 53, and a branch tube 54 is provided to allow water to go to waste when not required by the endoscope. Flow through the branch tube 54 is controlled by a needle valve 55 which can be open or closed.

While various embodiments of the present invention have been disclosed, it will be obvious to those skilled in the art that such embodiments are provided by way of example only. Further, each element or component of the present invention may be alternatively described as a means for performing the function or functions performed by the element or component. Numerous variations, changes, and substitutions will now occur to those skilled in the art without departing from the invention. Accordingly, it is intended that the invention be limited only by the spirit and scope of the appended claims.

1. An endoscope for introduction into and along a passageway in a human or animal body in a desired direction of travel, the endoscope comprising:
a fluid propulsion apparatus for effecting said introduction, the fluid propulsion apparatus comprising:
a conduit having an inlet for fluid under pressure and at least one outlet for expelling said fluid in a direction at least partly opposed to the desired direction of travel of the endoscope, and
at least one atomising nozzle.

2. An endoscope according to claim 1 wherein said conduit is movable lengthwise of the endoscope, but is constrained to substantially follow the path of the endoscope.

3. An endoscope for introduction into and along a passageway in a human or animal body in a desired direction of travel, the endoscope comprising:
a fluid propulsion apparatus, the fluid propulsion apparatus comprising a conduit having an inlet for fluid under pressure and at least one outlet for expelling said fluid in a direction at least partly opposed to the desired direction of travel of the endoscope, wherein said conduit is movable lengthwise of the endoscope, but is constrained to substantially follow the path of the endoscope.

4. An endoscope according to claim 3 wherein said conduit is movable lengthwise of the endoscope along a channel thereof.

5. An endoscope according to claim 4, wherein the channel is a biopsy channel.

6. A method of introducing an endoscope according to claim 2 into a patient a to a desired extent, the method comprising the steps of:
(a) moving the conduit a certain direction in the desired direction of travel;
(b) allowing endoscope to move in the desired direction; and
(c) repeating steps (a) and (b) until the endoscope has been introduced to the desired extent.

7. An endoscope according to 2 wherein the endoscope has a mass per unit length over a majority of its length of not more than about 1 gram/cm.

8. An endoscope according to claim 2 wherein the endoscope has a diameter of less than about 6 mm over a majority of its length.

9. An endoscope for introduction into and along a passageway in a human or animal body in a desired direction of travel, the endoscope having fluid propulsion apparatus comprising a fluid inlet and at least one outlet for expelling the fluid in a direction at least partly opposed to the desired direction of travel of the endoscope; wherein the endoscope has: (a) a mass per unit length over a majority of its length of not more than about 1 gram/cm; and (b) a diameter of less than about 6 mm over a majority of its length.

10. A method of introducing an endoscope into and along a passageway in a human body, the method comprising the steps of:
providing an endoscope;
providing a fluid propulsion apparatus associated with the endoscope;
providing a guidewire;
introducing the guidewire into the passageway in the body; and
advancing the endoscope along the guidewire by expelling fluid from the fluid propulsion apparatus in a direction at least partly opposed to the direction of travel.

11. The method of claim 10 wherein the step of advancing the endoscope along the guidewire comprises advancing the endoscope along a guidwire disposed exterior to a main body of the endoscope.

12. The method of claim 10 comprising providing a guidewire receiving element in the form of a tube having a length less than that of the length of the endoscope, and disposing the guidewire in the guidewire receiving element.

13. The method of claim 10 wherein the step of advancing the endoscope comprises advancing the endoscope into a naturally occurring passageway in the body.

13. The method of claim 10 wherein the step of advancing the endoscope comprises advancing the endoscope in a portion of the gastrointestinal tract of the body.

14. The method of claim 10 wherein the step of advancing the endoscope comprises advancing the endoscope in the colon.
